(19) United States
(12) Patent Application Publication
(10) Pub. No.: US 2014/0302050 A1
(43) Pub. Date: Oct. 9, 2014

(54) TARGETING AN AMPHIREGULIN-DERIVED CELL SURFACE NEO-EPITOPE

(71) Applicant: ALBERT EINSTEIN COLLEGE OF MEDICINE OF YESHIVA UNIVERSITY, Bronx, NY (US)

(72) Inventors: Paraic Anthony Kenny, Yonkers, NY (US); Kelly Susan Levano Najarro, Staten Island, NY (US); Eric Hoonee Jung, Exeter (GB)

(73) Assignee: ALBERT EINSTEIN COLLEGE OF MEDICINE OF YESHIVA UNIVERSITY, Bronx, NY (US)

(21) Appl. No.: 14/356,724

(22) PCT Filed: Oct. 24, 2012
(86) PCT No.: PCT/US12/61629

§ 371 Date: May 7, 2014

Related U.S. Application Data
(60) Provisional application No. 61/557,624, filed on Nov. 9, 2011.

(51) Int. Cl. C07K 16/22; A61K 35/14

(52) U.S. Cl. CPC C07K 16/22; A61K 35/15
USPC 424/139.1; 530/387.9; 530/387.3; 530/391.7; 530/391.3; 424/93.71

(57) ABSTRACT

Antibodies to an amphiregulin neo-epitope, fragments thereof, and compositions comprising such are provided. Therapies for cancers in which cells thereof express amphiregulin are provided, as well as assays for identifying additional agents useful in such therapies.

This application claims benefit of U.S. Provisional Application No. 61/557,624, filed Nov. 9, 2011, the contents of which are hereby incorporated by reference.

### BACKGROUND OF THE INVENTION

The disclosures of all publications, patents and patent application publications referred to in this application are hereby incorporated by reference in their entirety into the subject application to more fully describe the art to which the subject invention pertains.

Approximately 209,000 cases of breast cancer are diagnosed in the US each year. The majority of these tumors (˜70%) are Estrogen Receptor (ER) positive (ER+). Treatments blocking either the activity of the ER (Tamoxifen, Fulvestrant) or the production of estrogen itself (aromatase inhibitors) have led to significant increases in both disease-free and overall survival, however acquired resistance to endocrine therapy remains a significant problem. Accordingly, therapeutic targets in addition to ER and aromatase can have significant utility in treating this patient population. Moreover, standard-of-care treatments for women with endocrine-resistant breast cancer include various cytotoxic chemotherapies, but the long-term outcome in these cases is poor.

The present invention addresses the need for improved treatments and diagnoses for ER+ tumors, and identifies a novel epitope on ER+ tumor cells, and provides antibodies thereto and related therapies.

### SUMMARY OF THE INVENTION

This invention provides an isolated antibody, or an isolated fragment of an antibody, or an aptamer, which binds to a membrane-associated extracellular portion of a cleaved amphiregulin precursor protein.

A composition is also provided comprising the isolated antibody or the isolated fragment of an antibody or aptamer as described herein.

Also provided is a pharmaceutical composition comprising the isolated antibody or the isolated fragment of an antibody or aptamer as described herein.

Also provided is a method of treating a tumor in a subject comprising administering to the subject the antibody or antigen-binding fragment of an antibody or aptamer as described hereinabove, or the composition as described hereinabove, effective to treat the tumor.

Also provided is a method is provided of treating a tumor in a subject comprising administering to the subject an amount of dendritic cells loaded with cleaved amphiregulin precursor protein effective to treat a tumor.

Also provided is a method for identifying a candidate agent as an agent for treating a disease associated with expression of a cleaved amphiregulin precursor protein comprising contacting a membrane-associated extracellular portion of a cleaved amphiregulin precursor protein comprising SEQ ID NO:2 with the candidate agent and determining if the candidate agent binds thereto, wherein if the candidate agent binds thereto the candidate agent is identified as an agent for treating a disease associated with expression of cleaved amphiregulin precursor protein.

In addition, the invention provides an isolated antibody directed to a membrane-associated extracellular portion of a cleaved amphiregulin precursor protein or an isolated antigen-binding fragment thereof, or an aptamer directed to the cleaved amphiregulin precursor protein, for treatment of a tumor in a subject.

A method is also provided of identifying a tumor in a subject comprising administering to the subject a composition comprising an isolated antibody, or an isolated fragment of an antibody, or aptamer, which antibody or fragment or aptamer binds to a cleaved amphiregulin, conjugated to an imaging agent and detecting any bound antibody or fragment of an antibody or an aptamer conjugated to the imaging agent, thereby identifying a tumor in the subject.

Also provided is an isolated antibody or fragment of an antibody as described herein, wherein the antibody is, or the fragment is of, a human antibody, a humanized antibody or a chimeric antibody.

Compositions are also provided comprising any one or more of the isolated antibodies or the isolated fragments of an antibody described herein.

Pharmaceutical compositions are also provided comprising any one or more of the isolated antibodies or the isolated fragments of an antibody described herein.

Additional objects of the invention will be apparent from the description which follows.

### BRIEF DESCRIPTION OF THE DRAWINGS

FIG. 1A-1B: (1A). Amphiregulin expression levels in 295 tumors by microarray analysis, stratified by ER status. Y-axis is normalized log₂ gene expression level. Tumor data are from (van de Vijver et al., 2002). (1B). Amphiregulin expression levels in 13 Luminal breast cancer cell lines in 2D and 3D culture. Amphiregulin mRNA levels are well correlated with ERα mRNA levels (see Kenny et al., 2007).

FIG. 2: Western blot and coomassie blue stained membrane of Flag-tagged amphiregulin.

### DETAILED DESCRIPTION OF THE INVENTION

Abbreviations used herein:

Ab—antibody;
mAb—monoclonal antibody;
FcγR—Fc gamma receptor;
scFv—single-chain Fv;
CDR—complementarity determining region
Fab—antigen binding fragment (fragment antigen binding)
F(ab′)₂—antigen binding fragment comprising both Fab

In an embodiment, the amphiregulin is human amphiregulin. The sequence of the precursor protein of human amphiregulin is set forth GenBank: AAA51781.1:

(SEQ ID NO: 1)

| | |
|---|---|
| 1 | MRAPLLPPAP VVLSLLILGS GHYAAGLDLN DTYSGKREPF |
| | SGDHSADGFE VTSRSEMSSG |
| 61 | SEISPVSEMP SSSEPSSGAD YDYSEEYDNE PQIPGYIVDD |
| | SVRVEQVVKP PQNKTESENT |
| 121 | SDKPKRKKKG GKNGKNRRNR KKKNPCNAEF QNFCIHGECK |
| | YIEHLEAVTC KCQQEYFGER |
| 181 | CGEKSMKTHS MIDSSLSKIA LAAIAAFMSA VILTAVAVIT |
| | VQLRRQYVRK YEGEAEERKK |
| 241 | LRQENGNVHA IA |

The transmembrane sequence is residues 200 through 221 inclusive of SEQ ID NO:1. The N-terminus of protein is outside cell. The C-terminus of protein is inside cell. The neo-epitope is revealed by cleavage between K187 and T188, leaving an extracellular epitope, the amphiregulin neo-epitope, underlined above, of THSMIDSSLSKI (SEQ ID NO:2), i.e. residues 188 through 199 of SEQ ID NO:1. Thus, in an embodiment, a “cleaved” amphiregulin precursor protein would result from the cleavage of SEQ ID NO:1 between K187 and T188 thereof, with the C-terminal portion remaining associated with the cell membrane. A cleaved amphiregulin precursor protein comprises SEQ ID NO:2 but does not comprise SEQ ID NO:1. In an embodiment, the cleaved amphiregulin precursor protein consists of residues 188-252 of SEQ ID NO:1 and does not comprise residues 1-187 of SEQ ID NO:1.

As used herein, “membrane-associated” means physically or closely spatially associated with a portion of biomembrane, preferably a plasma membrane.

As used herein, “treating” a tumor means that one or more symptoms of the disease, such as the tumor itself, vascularization of the tumor, or other parameters by which the tumor is characterized, is or are reduced, ameliorated, prevented, placed in a state of remission, or maintained in a state of remission. “Treating” a tumor also means that one or more hallmarks of the tumor may be eliminated, reduced or prevented by the treatment.

In embodiments, the solid tumor is a tumor of the breast, colon, rectum, lung, nasopharynx, pharynx, bone, brain, sialaden, stomach, esophagus, testes, ovary, uterus, liver, small intestine, appendix, gall bladder, pancreas, kidney, urinary bladder, breast, cervix, vagina, vulva, prostate, testicle, thyroid or skin. In an embodiment, the solid tumor is an endocrine-resistant tumor. In an embodiment, the tumor is a tumor of the breast. In an embodiment, the tumor is ER+. In an embodiment, the tumor of the breast is ER+.

As used herein, “isolated” when describing an antibody or fragment means non-naturally occurring, produced by the hand of man.

This invention provides an isolated antibody, or an isolated fragment of an antibody, or aptamer which binds to a membrane-associated extracellular portion of a cleaved amphiregulin precursor protein. In an embodiment, the amphiregulin precursor protein is human. Cleaved refers to the natural proteolytic C-terminal processing of amphiregulin precursor protein in the body.

In an embodiment, the non-cleaved amphiregulin precursor protein comprises SEQ ID NO:1.

In an embodiment, the membrane-associated extracellular portion of the cleaved amphiregulin precursor protein does not comprise residues 1-187 of SEQ ID NO:1.

In an embodiment, the antibody binds an epitope comprising two or more residues of residues 188 to 199 as set forth in SEQ ID NO:1.

In an embodiment, the antibody binds a conformational epitope formed by residues 188 to 199 as set forth in SEQ ID NO:1.

In an embodiment, the antibody or aptamer binds SEQ ID NO:2 or a sequence comprising SEQ ID NO:2 but does not bind SEQ ID NO:1.

Also provided is an isolated antibody as described hereinabove, wherein the antibody is a human antibody, a humanized antibody or a chimeric antibody. In an embodiment, the antibody is a monoclonal antibody. In an embodiment, the antibody is a human antibody.

Also provided is an isolated fragment as described hereinabove, wherein the fragment is a fragment of a human antibody, of a humanized antibody or of a chimeric antibody. In an embodiment, the fragment is of a monoclonal antibody. In an embodiment, the fragment is of a human antibody. In an embodiment, the fragment comprises an Fab, an Fab′, an F(ab′)₂, an F_{d}, an Fᵥ, a complementarity determining region (CDR), or a single-chain antibody (scFv).

In an embodiment, the isolated antibody, or the isolated fragment of an antibody, is conjugated to a detectable agent or conjugated to a cytotoxic agent. (See, for example, Ducry L, Stump B. Antibody-drug conjugates: linking cytotoxic payloads to monoclonal antibodies. Bioconjug. Chem. 2010; 21:5-13, the contents of which are hereby incorporated by reference). In an embodiment, the cytotoxic agent is doxorubicin. In an embodiment, the cytotoxic agent is a maytansinoid. In a preferred embodiment, the cytotoxic agent an alkylating agent, an anti-metabolite, a plant alkaloid or terpenoid, or a cytotoxic antibiotic. In embodiments, the cytotoxic agent is cyclophosphamide, bleomycin, etoposide, platinum agent (cisplatin), fluorouracil, vincristine, methotrexate, taxol, epirubicin, leucovorin (folinic acid), or irinotecan. Detectable agents, for example imaging agents, comprise, but are not limited to moieties such as radionuclides, fluorescent dyes, chemiluminescent agents, microparticles, nanoparticles, enzymes, colorimetric labels, magnetic labels, haptens, molecular beacons and aptamer beacons. Such detectable agents can also comprise antibodies or antibody fragments to which the moieties listed herein are bound, conjugated or otherwise attached.

A composition is also provided comprising the isolated antibody or the isolated fragment of an antibody or the aptamer as described hereinabove.

Also provided is a pharmaceutical composition comprising the isolated antibody or the isolated fragment of an antibody or the aptamer as described hereinabove.

A method is provided of treating a tumor in a subject comprising administering to the subject the antibody or antigen-binding fragment of an antibody or aptamer as described hereinabove, or the composition as described hereinabove, effective to treat the tumor.

In an embodiment, the tumor is an ER+ tumor. In an embodiment, the tumor is a tumor of the breast. In an embodiment, the tumor is a tumor of the lung or is a colorectal tumor.

A method is provided of treating a tumor in a subject comprising administering to the subject an amount of dendritic cells loaded with cleaved amphiregulin precursor protein effective to treat a tumor. In an embodiment, the tumor is an ER+ tumor. In an embodiment, the tumor is a tumor of the breast. In an embodiment, the tumor is a tumor of the lung or is a colorectal tumor. In an embodiment, the dendritic cells loaded with cleaved amphiregulin precursor protein present the membrane-associated extracellular portion of cleaved amphiregulin precursor protein on their surface.

In an embodiment, the DCs are autologous DCs. In an embodiment, the DCs are loaded by being pulsed ex vivo with cleaved amphiregulin precursor protein prior to being administered to the subject. In an embodiment, the cleaved amphiregulin precursor protein is a membrane-associated extracellular portion of cleaved amphiregulin precursor protein. In an embodiment, the membrane-associated extracellular portion of cleaved amphiregulin precursor protein comprises SEQ ID NO:2. In an embodiment, the membrane-associated extracellular portion of cleaved amphiregulin precursor protein comprises residues 188 to 199 of SEQ ID NO:1.

A method is provided for identifying a candidate agent as an agent for treating a disease associated with expression of a cleaved amphiregulin precursor protein comprising contacting a membrane-associated extracellular portion of a cleaved amphiregulin precursor protein comprising SEQ ID NO:2 with the candidate agent and determining if the candidate agent binds thereto, wherein if the candidate agent binds thereto the candidate agent is identified as an agent for treating a disease associated with expression of cleaved amphiregulin precursor protein. In an embodiment, the method is not a diagnostic method and is not a treatment method.

In an embodiment, the candidate agent is an antibody, a fragment of an antibody, a peptide or an aptamer. In an embodiment, the candidate agent is a small molecule.

An isolated antibody is provided directed to a membrane-associated extracellular portion of a cleaved amphiregulin precursor protein or an isolated antigen-binding fragment thereof, or an aptamer directed to the cleaved amphiregulin precursor protein, for treatment of a tumor in a subject. In an embodiment, the antibody is, or the fragment is of, a human antibody, a humanized antibody or a chimeric antibody. In an embodiment, the antibody is a monoclonal antibody. In an embodiment, the antibody is a human antibody. In an embodiment, the antibody is a humanized antibody. In an embodiment, the tumor is an ER+ tumor. In an embodiment, the tumor is a tumor of the breast. In an embodiment, the tumor is a tumor of the lung or is a colorectal tumor.

A method of identifying a tumor in a subject comprising administering to the subject a composition comprising an isolated antibody, or an isolated fragment of an antibody, or an aptamer, which antibody or fragment or aptamer binds to a cleaved amphiregulin precursor protein and is conjugated to an imaging agent, and detecting any bound antibody or fragment of an antibody or aptamer conjugated to the imaging agent, thereby identifying a tumor in the subject. By detecting the imaging agent, the presence of the tumor is identified. The amount of imageable signal can be used to distinguish between cancerous and non-cancerous tissue and can also be used to stage the tumor. The detection step can occur at any suitable time after the imaging agent-conjugated antibody is administered. In an embodiment, the method can be used as a treatment in that the method can further comprise administering to a subject so-identified to have a tumor, one or more anti ER+ tumor medications so as to treat to the tumor.

In an embodiment, the tumor is an ER+ tumor. In an embodiment, the tumor is a tumor of the breast. In an embodiment, the tumor is a tumor of the lung or is a colorectal tumor. In an embodiment, the imaging agent is a radioactive imaging agent or a fluorescent imaging agent. In an embodiment, the composition comprises the isolated antibody or the isolated fragment of an antibody.

The invention provides an isolated antibody or fragment of an antibody as described hereinabove, wherein the antibody is a human antibody, a humanized antibody or a chimeric antibody. In an embodiment, the antibody is a monoclonal antibody. In an embodiment, the antibody is a human antibody. In an embodiment, the fragment is of a human antibody, of a humanized antibody or of a chimeric antibody. In an embodiment, the fragment is of a monoclonal antibody. In an embodiment, the fragment is of a human antibody. In an embodiment, the fragment comprises an Fab, an Fab′, an F(ab′)₂, an F_{d}, an Fᵥ, a complementarity determining region (CDR), or a single-chain antibody (scFv). In an embodiment, the fragment comprises a CDR3 of a Vₕ chain. In an embodiment the fragment also comprises one of, more than one of, or all of CDR1, CDR2 of Vₕ and CDR1, CDR2 and CDR3 of a Vₗ.

Compositions are also provided comprising any one or more of the isolated antibodies or the isolated fragments of an antibody or aptamers described herein.

Pharmaceutical compositions are also provided comprising any one or more of the isolated antibodies or the isolated fragments of an antibody or aptamers described herein.

As used herein, the term “antibody” refers to an intact antibody, i.e. with complete Fc and Fv regions. “Fragment” refers to any portion of an antibody, or portions of an antibody linked together, such as a single-chain Fv (scFv), which is less than the whole antibody but which is an antigen-binding portion and which competes with the intact antibody of which it is a fragment for specific binding. As such a fragment can be prepared, for example, by cleaving an intact antibody or by recombinant means. See generally, Fundamental Immunology, Ch. 7 (Paul, W., ed., 2nd ed. Raven Press, N.Y. (1989), hereby incorporated by reference in its entirety). Antigen-binding fragments may be produced by recombinant DNA techniques or by enzymatic or, for example, chemical cleavage of intact antibodies or by molecular biology techniques. In some embodiments, a fragment is an Fab, Fab′, F(ab′)₂, F_{d}, Fᵥ, complementarity determining region (CDR) fragment, single-chain antibody (scFv), (a variable domain light chain (V_{L}) and a variable domain heavy chain (V_{H}) linked via a peptide linker. In an embodiment the linker of the scFv is 10-25 amino acids in length. In an embodiment the peptide linker comprises glycine, serine and/or threonine residues. For example, see Bird et al., Science, 242: 423-426 (1988) and Huston et al., Proc. Natl. Acad. Sci. USA, 85:5879-5883 (1988) each of which are hereby incorporated by reference in their entirety), or a polypeptide that contains at least a portion of an antibody that is sufficient to confer amphiregulin neo-epitope-specific antigen binding on the polypeptide, including a diabody. From N-terminus to C-terminus, both the mature light and heavy chain variable domains comprise the regions FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987), or Chothia et al., Nature 342:878-883 (1989), each of which are hereby incorporated by reference in their entirety). As used herein, the term “polypeptide” encompasses native or artificial proteins, protein fragments and polypeptide analogs of a protein sequence. A polypeptide may be monomeric or polymeric. As used herein, an Fₐ fragment means an antibody fragment that consists of the V_{H} and CH1 domains; an Fᵥ fragment consists of the V₁ and V_{H} domains of a single arm of an antibody; and a dAb fragment (Ward et al., Nature 341:544-546 (1989) hereby incorporated by reference in its entirety) consists of a V_{H} domain.

In some embodiments, fragments are at least 5, 6, 8 or 10 amino acids long. In other embodiments, the fragments are at least 14, at least 20, at least 50, or at least 70, 80, 90, 100, 150 or 200 amino acids long.

The term “monoclonal antibody” is not intended, unless otherwise indicated, to be limited as regards to the source of the antibody or the manner in which it is made (e.g., by hybridoma, phage selection, recombinant expression, transgenic animals, etc.). The term “monoclonal antibody” as used herein refers to an antibody member of a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. Thus an identified monoclonal antibody can be produced by non-hybridoma techniques, e.g. by appropriate recombinant means once the sequence thereof is identified. In one embodiment, however, monoclonal means as produced by a single type of hybridoma.

In an embodiment the composition or pharmaceutical composition comprising one or more of the antibodies or fragments described herein is substantially pure with regard to the antibody or fragment. A composition or pharmaceutical composition comprising one or more of the antibodies or fragments described herein is “substantially pure” with regard to the antibody or fragment when at least about 60 to 75% of a sample of the composition or pharmaceutical composition exhibits a single species of the antibody or fragment. A substantially pure composition or pharmaceutical composition comprising one or more of the antibodies or fragments described herein can comprise, in the portion thereof which is the antibody or fragment, 60%, 70%, 80% or 90% of the antibody or fragment of the single species, more usually about 95%, and preferably over 99%. Antibody purity or homogeneity may tested by a number of means well known in the art, such as polyacrylamide gel electrophoresis or HPLC.

As used herein, a “human antibody” unless otherwise indicated is one whose sequences correspond to (i.e. are identical in sequence to) an antibody that could be produced by a human and/or has been made using any of the techniques for making human antibodies as disclosed herein. This definition of a human antibody specifically excludes a humanized antibody and also excludes an antibody actually made in a human. A “human antibody” as used herein can be produced using various techniques known in the art, including phage-display libraries (e.g. Hoogenboom and Winter, J. Mol. Biol., 227:381 (1991); Marks et al., J. Mol. Biol., 222:581 (1991), hereby incorporated by reference in its entirety), by methods described in Cole et al., Monoclonal Antibodies and Cancer Therapy, Alan R. Liss, p. 77 (1985) (hereby incorporated by reference in its entirety); Boerner et al., J. Immunol., 147(1):86-95 (1991) (hereby incorporated by reference in its entirety), van Dijk and van de Winkel, Curr. Opin. Pharmacol., 5: 368-74 (2001) (hereby incorporated by reference in its entirety), and by administering the antigen (e.g. cleaved amphiregulin, or cleaved amphiregulin in membrane portion) to a transgenic animal that has been modified to produce such antibodies in response to antigenic challenge, but whose endogenous loci have been disabled, e.g., immunized xenomice (see, e.g., U.S. Pat. Nos. 5,939,598; 6,075,181; 6,114,598; 6,150,584 and 6,162,963 to Kucherlapati et al. regarding XENOMOUSE™ technology, each of which patents are hereby incorporated by reference in their entirety), e.g. Veloclmmune® (Regeneron, Tarrytown, N.Y.), e.g. UltiMab® platform (Medarex, now Bristol Myers Squibb, Princeton, N.J.). See also, for example, Li et al., Proc. Natl. Acad. Sci. USA, 103:3557-3562 (2006) regarding human antibodies generated via a human B-cell hybridoma technology. See also KM Mouse® system, described in PCT Publication WO 02/43478 by Ishida et al., in which the mouse carries a human heavy chain transchromosome and a human light chain transgene, and the TC mouse system, described in Tomizuka et al. (2000) Proc. Natl. Acad. Sci. USA 97:722-727, in which the mouse carries both a human heavy chain transchromosome and a human light chain transchromosome, both of which are hereby incorporated by reference in their entirety. In each of these systems, the transgenes and/or transchromosomes carried by the mice comprise human immunoglobulin variable and constant region sequences.

The term “human antibody”, as used herein, is intended to include antibodies having variable regions in which both the framework and CDR regions are sequences of human origin or identical thereto. Furthermore, if the antibody (e.g. an intact antibody rather than, for example, an Fab fragment) contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. In one non-limiting embodiment, where the human antibodies are human monoclonal antibodies, such antibodies can be produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from a non-human animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region (HVR) of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin variable domain are replaced by corresponding non-human residues. These modifications may be made to further refine antibody performance. Furthermore, in a specific embodiment, humanized antibodies may comprise residues that are not found in the recipient antibody or in the donor antibody. In an embodiment, the humanized antibodies do not comprise residues that are not found in the recipient antibody or in the donor antibody. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. See, e.g., Jones et al., Nature 321:522-525 (1986); Riechmann et al., Nature 332:323-329 (1988); Presta, Curr. Op. Struct. Biol. 2:593-596 (1992); Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409, the contents of each of which references and patents are hereby incorporated by reference in their entirety. In one embodiment where the humanized antibodies do comprise residues that are not found in the recipient antibody or in the donor antibody, the Fc regions of the antibodies are modified as described in WO 99/58572, the content of which is hereby incorporated by reference in its entirety.

Techniques to humanize a monoclonal antibody are described in U.S. Pat. Nos. 4,816,567; 5,807,715; 5,866,692; 6,331,415; 5,530,101; 5,693,761; 5,693,762; 5,585,089; and 6,180,370, the content of each of which is hereby incorporated by reference in its entirety.

A number of “humanized” antibody molecules comprising an antigen-binding site derived from a non-human immunoglobulin have been described, including antibodies having rodent or modified rodent V regions and their associated complementarity determining regions (CDRs) fused to human constant domains. See, for example, Winter et al. Nature 349: 293-299 (1991), Lobuglio et al. Proc. Nat. Acad. Sci. USA 86: 4220-4224 (1989), Shaw et al. J. Immunol. 138: 4534-4538 (1987), and Brown et al. Cancer Res. 47: 3577-3583 (1987), the content of each of which is hereby incorporated by reference in its entirety. Other references describe rodent hypervariable regions or CDRs grafted into a human supporting framework region (FR) prior to fusion with an appropriate human antibody constant domain. See, for example, Riechmann et al. Nature 332: 323-327 (1988), Verhoeyen et al. Science 239: 1534-1536 (1988), and Jones et al. Nature 321: 522-525 (1986), the content of each of which is hereby incorporated by reference in its entirety. Another reference describes rodent CDRs supported by recombinantly veneered rodent framework regions—European Patent Publication No. 0519596 (incorporated by reference in its entirety). These “humanized” molecules are designed to minimize unwanted immunological response toward rodent anti-human antibody molecules which limits the duration and effectiveness of therapeutic applications of those moieties in human recipients. The antibody constant region can be engineered such that it is immunologically inert (e.g., does not trigger complement lysis). See, e.g. PCT Publication No. WO99/58572; UK Patent Application No. 9809951.8. Other methods of humanizing antibodies that may also be utilized are disclosed by Daugherty et al., Nucl. Acids Res. 19: 2471-2476 (1991) and in U.S. Pat. Nos. 6,180,377; 6,054,297; 5,997,867; 5,866,692; 6,210,671; and 6,350,861; and in PCT Publication No. WO 01/27160 (each incorporated by reference in their entirety).

Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

In embodiments, the antibodies or fragments herein can be produced recombinantly, for example antibodies expressed using a recombinant expression vector transfected into a host cell, antibodies isolated from a recombinant, combinatorial human antibody library, antibodies isolated from a non-human animal (e.g., a mouse) that is transgenic for human immunoglobulin genes.

In an embodiment the antibody fragment specifically binds to SEQ ID NO:2, or to a polypeptide which comprises SEQ ID NO:2 but which does not have the sequence set forth in SEQ ID NO: 1. As used herein, the terms “is capable of specifically binding”, “specifically binds”, or “preferentially binds” refers to the property of an antibody or fragment of binding to the (specified) antigen with a dissociation constant that is <1 μM, preferably <1 nM and most preferably <10 pM. In an embodiment, the K_{d} of the antibody for the amphiregulin neo-epitope (i.e. comprising the sequence set forth in SEQ ID NO:2) is 250-500 pM. An epitope that “specifically binds”, or “preferentially binds” (used interchangeably herein) to an antibody or a polypeptide is a term well understood in the art, and methods to determine such specific or preferential binding are also well known in the art. A molecular entity is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to amphiregulin neo-epitope is an antibody that binds this epitope with greater affinity, avidity, more readily, and/or with greater duration than it binds to non-amphiregulin epitopes. It is also understood that an antibody (or moiety or epitope) that specifically or preferentially binds to a first target may or may not specifically or preferentially bind to a second target. As such, “specific binding” or “preferential binding” does not necessarily require (although it can include) exclusive binding. In an embodiment, the antibody or fragment binds to the amphiregulin target.

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

Depending on the amino acid sequences of the constant domains of their heavy chains, antibodies (immunoglobulins) can be assigned to different classes. The antibody or fragment can be, e.g., any of an IgG, IgD, IgE, IgA or IgM antibody or fragment thereof, respectively. In an embodiment the antibody is an immunoglobulin G. In an embodiment the antibody fragment is a fragment of an immunoglobulin G. In an embodiment the antibody is an IgG1, IgG2, IgG2a, IgG2b, IgG3 or IgG4. In an embodiment the antibody comprises sequences from a human IgG1, human IgG2, human IgG2a, human IgG2b, human IgG3 or human IgG4. A combination of any of these antibodies subtypes can also be used. One consideration in selecting the type of antibody to be used is the desired serum half-life of the antibody. For example, an IgG generally has a serum half-life of 23 days, IgA 6 days, IgM 5 days, IgD 3 days, and IgE 2 days. (Abbas A K, Lichtman A H, Pober J S. Cellular and Molecular Immunology, 4th edition, W.B. Saunders Co., Philadelphia, 2000, hereby incorporated by reference in its entirety).

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (K) and lambda (4 based on the amino acid sequences of their constant domains.

“Framework” or “FR” residues are those variable domain residues other than the HVR residues as herein defined.

The term “hypervariable region” or “HVR” when used herein refers to the regions of an antibody variable domain which are hypervariable in sequence and/or form structurally defined loops. Generally, antibodies comprise six HVRs; three in the V_{H} (H1, H2, H3) and three in the V_{L} (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996). A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991) hereby incorporated by reference in its entirety). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the V_{H}. The variable domain residues are numbered according to Kabat et al., supra, for each of these definitions.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine of the Fc region may be removed, for example, during production or purification of the antibody, or by recombinantly engineering the nucleic acid encoding a heavy chain of the antibody. Accordingly, an intact antibody as used herein may be an antibody with or without the otherwise C-terminal cysteine.

As used herein a “conformational” amphiregulin neo-epitope is an epitope formed by a plurality of amino acids, at least two of which are discontinuous, arranged in a three-dimensional conformation due to the native folding of the antigen. The conformational epitope is recognized by the antigen-binding portion of an antibody directed to the conformational epitope. In an embodiment, the conformational epitope is formed by the residues of SEQ ID NO:2.

Compositions or pharmaceutical compositions comprising the antibodies, ScFvs or fragments of antibodies disclosed herein are preferably comprise stabilizers to prevent loss of activity or structural integrity of the protein due to the effects of denaturation, oxidation or aggregation over a period of time during storage and transportation prior to use. The compositions or pharmaceutical compositions can comprise one or more of any combination of salts, surfactants, pH and tonicity agents such as sugars can contribute to overcoming aggregation problems. Where a composition or pharmaceutical composition of the present invention is used as an injection, it is desirable to have a pH value in an approximately neutral pH range, it is also advantageous to minimize surfactant levels to avoid bubbles in the formulation which are detrimental for injection into subjects. In an embodiment, the composition or pharmaceutical composition is in liquid form and stably supports high concentrations of bioactive antibody in solution and is suitable for parenteral administration, including intravenous, intramuscular, intraperitoneal, intradermal and/or subcutaneous injection. In an embodiment, the composition or pharmaceutical composition is in liquid form and has minimized risk of bubble formation and anaphylactoid side effects. In an embodiment, the composition or pharmaceutical composition is isotonic. In an embodiment, the composition or pharmaceutical composition has a pH or 6.8 to 7.4.

In an embodiment the ScFvs or fragments of antibodies disclosed herein are lyophilized and/or freeze dried and are reconstituted for use.

Examples of pharmaceutically acceptable carriers include, but are not limited to, carriers comprising phosphate buffered saline solution, sterile water (including water for injection USP), emulsions such as oil/water emulsion, and various types of wetting agents. Preferred diluents for aerosol or parenteral administration are phosphate buffered saline or normal (0.9%) saline, for example 0.9% sodium chloride solution, USP. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, the content of each of which is hereby incorporated in its entirety). In non-limiting examples, the can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxy)ethoxy]ethyl (E)-octadec-9-enoate), disodium edetate dehydrate, sucrose, monobasic sodium phosphate monohydrate, and dibasic sodium phosphate dihydrate.

The antibodies, or fragments of antibodies, or compositions, or pharmaceutical compositions described herein can also be lyophilized or provided in any suitable forms including, but not limited to, injectable solutions or inhalable solutions, gel forms and tablet forms.

The term “K_{d}”, as used herein, is intended to refer to the dissociation constant of an antibody-antigen interaction. One way of determining the K_{d} or binding affinity of antibodies to amphiregulin neo-epitopes is by measuring binding affinity of monofunctional Fab fragments of the antibody. (The affinity constant is the inverted dissociation constant). To obtain monofunctional Fab fragments, an antibody (for example, IgG) can be cleaved with papain or expressed recombinantly. The affinity of an anti-amphiregulin neo-epitope Fab fragment of an antibody can be determined by surface plasmon resonance (BIAcore3000™ surface plasmon resonance (SPR) system, BIAcore Inc., Piscataway N.J.). CMS chips can be activated with N-ethyl-N′-(3-dimethylaminopropyl)-carbodiinide hydrochloride (EDC) and N-hydroxysuccinimide (NHS) according to the supplier's instructions. The amphiregulin neo-epitope can be diluted into 10 mM sodium acetate pH 4.0 and injected over the activated chip at a concentration of 0.005 mg/mL. Using variable flow time across the individual chip channels, two ranges of antigen density can be achieved: 100-200 response units (RU) for detailed kinetic studies and 500-600 RU for screening assays. Serial dilutions (0.1-10× estimated K_{d}) of purified Fab samples are injected for 1 min at 100 microliters/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ and dissociation rates (k_{off}) are obtained simultaneously by fitting the data to a 1:1 Langmuir binding model (Karlsson, R. Roos, H. Fagerstam, L. Petersson, B. (1994). Methods Enzymology 6. 99-110, the content of which is hereby incorporated in its entirety) using the BIA evaluation program. Equilibrium dissociation constant (K_{d}) values are calculated as k_{off}/kₒₙ. This protocol is suitable for use in determining binding affinity of an antibody or fragment to any amphiregulin neo-epitope. Other protocols known in the art may also be used. For example, ELISA of amphiregulin neo-epitope with mAb can be used to determine the k_{D} values.

The invention also provides aptamers to the cleaved amphiregulin target and uses thereof. Aptamers are single stranded oligonucleotides or oligonucleotide analogs that bind to a particular target molecule. Aptamers are smaller than antibodies, generally in the range of 50-100 nt. Their binding is highly dependent on the secondary structure formed by the aptamer oligonucleotide. Both RNA and single-stranded DNA (or analog) aptamers are known. See, e.g., U.S. Pat. Nos. 5,773,598; 5,496,938; 5,580,737; 5,654,151; 5,726,017; 5,786,462; 5,503,978; 6,028,186; 6,110,900; 6,124,449; 6,127,119; 6,140,490; 6,147,204; 6,168,778; and 6,171,795. Aptamers can also be administered already synthesized or expressed from a transfected vector (Joshi et al., 2002, J. Virol. 76, 6545). Aptamers to the cleaved amphiregulin are readily identified. Aptamers can be selected by using an iterative process called SELEX (systematic Evolution of Ligands by EXponential enrichment) (see, e.g., Burke et al., 1996., J. Mol. Biol. 264, 650; Ellington and Szostak, 1990, Nature 346, 818; Schneider et al., 1995, Biochemistry 34, 9599; Tuerk and Gold, 1992, Proc. Natl. Acad. Sci. USA 89:6988; Tuerk and Gold, 1990, Science 249:505). Several variations of SELEX have been developed which improve the process and allow its use under particular circumstances. See, e.g., U.S. Pat. Nos. 5,472,841; 5,503,978; 5,567,588; 5,582,981; 5,637,459; 5,683,867; 5,705,337; 5,712,375; and 6,083,696.

As used herein, the term “subject” for purposes of treatment includes any subject, and preferably is a subject who is in need of the treatment of the targeted pathologic condition for example an amphiregulin neo-epitope-associated pathology such as an ER+ cancer. The term “subject” is intended to include living mammals, e.g., humans, dogs, cows, horses, pigs, sheep, goats, cats, mice, rabbits, rats, and transgenic non-human animals. In specific embodiments of the invention, the subject is a human.

As used herein a “small molecule” is an organic compound either synthesized in the laboratory or found in nature which contains carbon-carbon bonds, and has a molecular mass of less than 2000 daltons. The small molecule may be a substituted hydrocarbon or an un-substituted hydrocarbon.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

Disclosed herein is a structural feature commonly found in ER+ breast tumors. This structure provides a unique way of targeting suitable therapies to greatly enrich their concentration at the tumor site while sparing cancer-free organs. The structural feature is the cleaved stalk of amphiregulin. There are 209,000 cases of breast cancer in the US each year, approximately ⅔ of which are likely to express the cleaved stalk of amphiregulin. Amphiregulin expression has also been reported at high levels in lung and colorectal tumors and there are additional solid tumors in which a therapeutic approach based on this structural feature is also relevant.

### Materials, Methods and Results

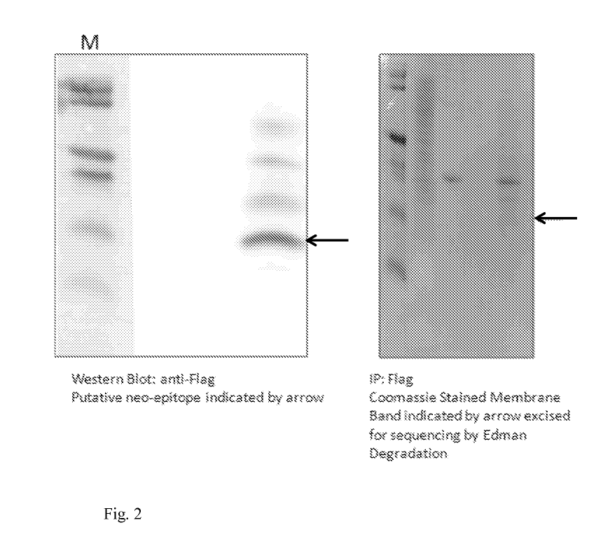

A C-terminal Flag-tagged human amphiregulin cDNA was generated. This cDNA was overexpressed in HEK293 cells. Western blot analysis shows that amphiregulin is processed in multiple stages (FIG. 2, left). The smallest fragment which would correspond to the transmembrane stalk was sought. The Flag-tagged protein was immunopurified, transferred to a membrane (FIG. 2, right), the band excised and submitted for Edman Degradation sequencing. Sequencing data was obtained for seven cycles, which identified the first, second, third, fourth, fifth and seventh amino acids of the protein fragment. The sixth amino acid was not successfully identified. The sequence provided was THSMxDS. The only THSMxDS motif in amphiregulin, however, is found in the extracellular juxtamembrane region identified above in the Detailed Description.

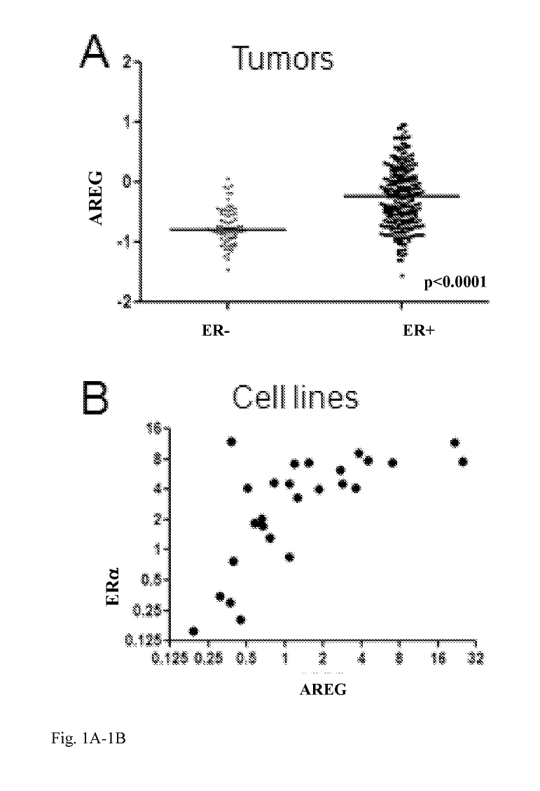

In breast cancer, amphiregulin expression is most commonly found in ERpositive (ER+) breast cancers (FIG. 1A), a finding that is also observed in breast cancer cell lines (FIG. 1B). In the patients cohort shown in FIG. 1A, the overall survival for ER+ breast cancer patients at ten years was 78%, while the overall survival of the patients in the highest quartile of amphiregulin expression was 85% at ten years. Among ER+ tumors, amphiregulin expression appears to be enriched in lower grade tumors which tend to have a good outcome, although 15% of these patients still died within ten years. These data are from primary tumors at the time of surgery. It is proposed here that Amphiregulin continues to be important later, in the 40% of ER tumors that progress to endocrine resistance. In two out of three cell line models examined so far, amphiregulin expression is maintained at high levels. Analysis of microarray data from other studies (e.g. NCBI GEO GSE14513) indicates that maintenance of amphiregulin expression is a frequent characteristic of many endocrine-resistant lines. Accordingly, the transmembrane stalk targeted herein likely continues to be present in a significant proportion of endocrine-resistant tumors (for which the prognosis is poor in most cases), and the treatment strategy also has clinical applicability in that setting.

Cell surface biotinylation experiments were performed at defined time points after treatment with a TACE inhibitor (which prevents amphiregulin cleavage and generation of new stalks). Initially it was thought that the stalks would be rapidly internalized and experiments were performed for a very short time course up to ten minutes. However, at ten minutes, there was still substantial amounts of amphiregulin stalk at the cell surface available for biotinylation (detected by neutravidin IP, followed by Western blot). Subsequent experiments demonstrated that cell-surface amphiregulin stalk was still detectable after 240 minutes.

An antibody to the extracellular N-terminal portion of cleavage product can be characterized by comparing its binding affinity to several breast cancer cell lines in which this laboratory has already determined the level of the target (high levels: MCF7, T47D, BT474; low levels BT549, MDA-MB-468, Hs578T). Sensitivity of cell lines expressing high and low levels of the target can be evaluated in vitro by performing dose/response cytotoxicity assays. Sensitivity to ADCC can be determined by co-culture of the breast cancer cell lines with human peripheral blood mononuclear cells.

Xenografts of cell lines expressing high and low levels of the target established in SCID mice permits treatment with the antibody to characterize anti-tumor efficacy in vivo.

### Discussion

Herein, it is disclosed that a particular cell-surface protein, amphiregulin, is very highly expressed in the majority of ER+ breast cancers. The gene encoding this protein is a transcriptional target of the ER. The protein is synthesized as a transmembrane precursor protein which is activated by proteolytic cleavage to release an soluble signaling domain which can interact with a cell-surface receptor and elicit oncogenic signaling. The transmembrane stalk left behind after the release of the active signaling component of this protein has received little attention. The N-terminal amino acid sequence of this transmembrane stalk (which is usually concealed and inaccessible in the full-length protein) functions as a neo-epitope against which therapies can be targeted. Antibodies, aptamers and other agents which recognize this region will have therapeutic efficacy in breast, lung, colorectal and other solid tumors that express high levels of the target. The agent can be used alone (e.g. an antibody which recruits an immune response and kills target cells via antigen-dependent cellular cytotoxicity) or with a therapeutic cargo (e.g. doxorubicin or a maytansinoid) conjugated to the agent to significantly elevate the levels of that therapeutic in the vicinity of the cancer cells and/or to promote the internalization of the therapeutic cargo by the cancer cell.

In addition, because the agent can be labeled with a fluorescent or other detectable marker, it can be used to interrogate tissue specimens via immunohistochemistry or other means to identify specimens expressing high levels of the target. Such an approach is useful as a predictive biomarker in selecting appropriate cohorts of patients for treatment with therapies utilizing the agent.

### REFERENCES

Kenny, P. A. (2007). Tackling EGFR signaling with TACE antagonists: a rational target for metalloprotease inhibitors in cancer. Expert Opin Ther Targets 11, 1287-1298.
Kenny, P. A., and Bissell, M. J. (2007a). Targeting TACE-dependent EGFR ligand shedding in breast cancer. J Clin Invest 117, 337-345.
Kenny, P. A., and Bissell, M. J. (2007b). Targeting TNF-Alpha converting enzyme (TACE)-dependent growth factor shedding in cancer therapy. In United States Patent Application Publication US 2009/0274626 A1 (The Regents of the University of California).
Kenny, P. A., Lee, G. Y., Myers, C. A., Neve, R. M., Semeiks, J. R., Spellman, P. T., Lorenz, K., Lee, E. H., Barcellos-Hoff, M. H., Petersen, O. W., et al. (2007). The morphologies of breast cancer cell lines in three-dimensional assays correlate with their profiles of gene expression. Molecular Oncology 1, 84-96.
van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009.

What is claimed is:

1. An isolated antibody, or an isolated fragment of an antibody, or an aptamer, which binds to a membrane-associated extracellular portion of a cleaved amphiregulin precursor protein.

2. The isolated antibody, or isolated fragment of an antibody, or aptamer of claim 1, wherein the non-cleaved amphiregulin precursor protein comprises SEQ ID NO:1.

3. The isolated antibody, or isolated fragment of an antibody, or aptamer of claim 1, wherein the membrane-associated extracellular portion of the cleaved amphiregulin precursor protein does not comprise residues 1-187 of SEQ ID NO:1.

4. The isolated antibody, or isolated fragment of an antibody, or aptamer of claim 1, wherein the antibody binds an epitope comprising two or more residues of residues 188 to 199 as set forth in SEQ ID NO:1.

5. The isolated antibody, or isolated fragment of an antibody, or aptamer, of claim 1, wherein the antibody binds a conformational epitope formed by two or more residues of residues 188 to 199 as set forth in SEQ ID NO:1.

6. The isolated antibody, or isolated fragment of an antibody, or aptamer, of claim 1, wherein the antibody or aptamer binds SEQ ID NO:2 but does not bind SEQ ID NO:1.

7. An isolated antibody of claim 1, wherein the antibody is a human antibody, a humanized antibody or a chimeric antibody.

8. The isolated antibody of claim 7, wherein the antibody is a monoclonal antibody.

9. The isolated antibody of claim 7, wherein the antibody is a human antibody.

10. An isolated fragment of an antibody of claim 1, wherein the fragment is a fragment of a human antibody, of a humanized antibody or of a chimeric antibody.

11. The isolated fragment of an antibody of claim 10, wherein the fragment is of a monoclonal antibody.

12. The isolated fragment of an antibody of claim 10, wherein the fragment is of a human antibody.

13. The isolated fragment of an antibody of claim 10, wherein the fragment comprises an Fab, an Fab′, an F(ab′)₂, an F_{d}, an Fᵥ, a complementarity determining region (CDR), or a single-chain antibody (scFv).

14. The isolated antibody, or isolated fragment of an antibody, of claim 10 conjugated to a detectable agent or conjugated to a cytotoxic agent.

15. A composition comprising the isolated antibody of claim 1, or an isolated fragment thereof of claim 1.

16. A pharmaceutical composition comprising the isolated antibody of claim 1, or an isolated fragment thereof.

17. A method of treating a solid tumor in a subject comprising administering to the subject the antibody, or an antigen-binding fragment thereof, or aptamer of claim 1 effective to treat the solid tumor.

18. The method of claim 17, wherein the tumor is an ER+ tumor.

19. (canceled)

20. The method of claim 17, wherein the tumor is a tumor of the lung or is a colorectal tumor.

21-38. (canceled)

39. A method is provided of treating a tumor in a subject comprising administering to the subject an amount of dendritic cells loaded with cleaved amphiregulin precursor protein effective to treat a tumor.


SEQUENCE LISTING (2 sequences)

SEQ ID NO: 1 - protein, 252 aa, homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 2 - protein, 12 aa, homo sapiens
THSMIDSSLSKI